(19) United States
(12) Patent Application Publication
(10) Pub. No.: US 2020/0357490 A1
(43) Pub. Date: Nov. 12, 2020

(54) SYSTEM FOR CREATING A VIRTUAL CLINICAL TRIAL FROM ELECTRONIC MEDICAL RECORDS

(71) Applicant: International Business Machines Corporation, Armonk, NY (US)

(72) Inventors: Uri Kartoun, Cambridge, MA (US); Ping Zhang, White Plains, NY (US); Yoonyoung Park, Cambridge, MA (US); Kenney Ng, Arlington, MA (US); Fang Lu, Billerica, MA (US); Michael J. LaScaleia, Sudbury, MA (US); Francis Campion, Westwood, MA (US)

(21) Appl. No.: 16/405,025

(22) Filed: May 7, 2019

(51) Int. Cl. G16H 10/20; G16H 10/60; G06F 17/28

(52) U.S. Cl. CPC G16H 10/20; G06F 17/28; G16H 10/60

(57) ABSTRACT

A data analysis system is configured for alerting to the results of a virtual clinical trial. The data analysis system has an extraction system configured to extract a correlation between an ingredient and an effect from a clinical trial document using natural language processing, and extract medical history information for a population exposed to the ingredient and a population unexposed to the ingredient from stored electronic medical records. The data analysis system also has a virtual trial system configured to perform statistical analysis on the medical history information to assess the level of association between the ingredient and the effect, a decision system configured to determine an accuracy score for the correlation based on the statistical analysis, and an alerting system configured to provide the accuracy score to a user interface of an end-user device.

### TECHNICAL FIELD

The present application relates generally to generating results from electronic medical records (EMRs) and, more particularly, to a system for creating a virtual clinical trial from EMRs.

### BACKGROUND

Clinical trials are of vital importance to the development of pharmaceutical and medical products. Clinical trials that report on the level of association between taking an ingredient and experiencing adverse effects are limited to selected small populations. It could be that the same ingredient may have no effect if tested on additional independent populations. Alternatively, such effects may be more impactful on other populations. The limitations of clinical trials may result in insufficient or unrepresentative data for ingredient effects. Over the past several decades processing techniques have progressed such that large databases of medical history information are available for rapid data analysis. Many publications describe how applying computational methods on such databases can identify the effect of certain ingredients and treatments for a selected population. This information, achieved independently of the actual clinical trials, can be useful in evaluating association effects of medical treatments and pharmaceuticals and assessing the conclusions of clinical trials.

### SUMMARY

In some embodiments, a computer-implemented method for alerting to the results of virtual clinical trial in a data processing system is comprising a processing device and a memory comprising instructions which are executed by the processor is disclosed. The method includes extracting data from a clinical trial document, the clinical trial document including results of a clinical trial and including an ingredient, extracting data from a medical records database based on the ingredient, the data including medical history information for a plurality of patients, performing a virtual clinical trial on the medical history information from the plurality of patients, assessing the results of the virtual clinical trial, including determining a result associated with a correlation involving the ingredient, and alerting to the result by providing information to a user interface of an end-user device.

In other embodiments, a data analysis system for alerting to the results of a virtual clinical trial is disclosed. The data analysis system includes an extraction system configured to extract a correlation between an ingredient and an effect from a clinical trial document using natural language processing, and extract medical history information for a population exposed to the ingredient and a population unexposed to the ingredient from stored electronic medical records. The data analysis system also includes a virtual trial system configured to perform statistical analysis on the medical history information to assess the level of association between the ingredient and the effect, a decision system configured to determine an accuracy score for the correlation based on the statistical analysis, and an alerting system configured to provide the accuracy score to a user interface of an end-user device.

### BRIEF DESCRIPTION OF THE DRAWINGS

The foregoing and other aspects of the present invention are best understood from the following detailed description when read in connection with the accompanying drawings. For the purpose of illustrating the invention, there is shown in the drawings embodiments that are presently preferred, it being understood, however, that the invention is not limited to the specific instrumentalities disclosed. Included in the drawings are the following Figures:

FIG. 1 depicts a block diagram of an exemplary healthcare data environment, consistent with disclosed embodiments;

FIG. 2 is a block diagram of an example data processing system in which aspects of the illustrative embodiments are implemented;

FIG. 3 is a block diagram of an exemplary data analysis system, consistent with disclosed embodiments;

FIG. 4 is an example of a user interface for displaying a clinical trial result, consistent with disclosed embodiments;

FIG. 5 is an example of a depiction of medical history information for a pair of patients, consistent with disclosed embodiments; and

FIG. 6 is a flowchart of an exemplary process for alerting to the result of an virtual clinical trial, consistent with disclosed embodiments; and

FIG. 7 is an example of a user interface for displaying a table of clinical trial results together with a result from a virtual clinical trial, consistent with disclosed embodiments.

### DETAILED DESCRIPTION OF EXEMPLARY EMBODIMENTS

Embodiments of the present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a head disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network (LAN), a wide area network (WAN) and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including LAN or WAN, or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operations steps to be performed on the computer, other programmable apparatus, or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical functions. In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The present disclosure relates to the use of EMRs and the medical data that is stored therein. EMRs may be a compilation of all information that has been recorded and stored in one or more locations in relation to a patient. An example EMR may contain demographic information, allergies, diagnoses, vital sign information, medications prescribed and taken, laboratory tests conducted and the results, operations, providers and physicians visited, physical examination records, pathology reports, clinical narrative notes, discharge summaries, radiology reports, cardiology reports, and encounter information. The medical data may be a representation of a patient's medical history stored in an EMR.

The EMR may be organized or unorganized and contained structured and/or unstructured data. An organized EMR may contain metadata and categorized information that indicates that a software program can identify the stored information. An unorganized EMR may contain the information without software being able to identify what the information represents. Structured data may be tables, completed forms, test results, etc. that are easily recognizable and extractable as medical data. Unstructured data may include notes such as clinical narrative notes.

The present disclosure relates to a system capable of extracting content reported by clinical trials, and assessing each outcome on independent populations using EMRs. The system creates association models, such as a model that takes into account the effect as an outcome, identifies covariates, and calculates levels of association in univariate and adjusted models. The system can be used to support future clinical trials, or propose further investigating for the effects of an ingredient not yet reported in clinical trials or in the scientific literature.

Moreover, the present disclosure relates to a system that may be used to perform virtual clinical trials to test associations for a desired population subset. For example, the disclosed system may receive direct requests for data analysis and perform a virtual clinical trial to determine any relevant correlations between an ingredient and an effect. Further, the use of techniques such as natural language processing (NLP) and machine learning algorithms to enhance EMRs provides a clearer picture of medical history information for patients and provides a more effective pool of data for virtual clinical trials.

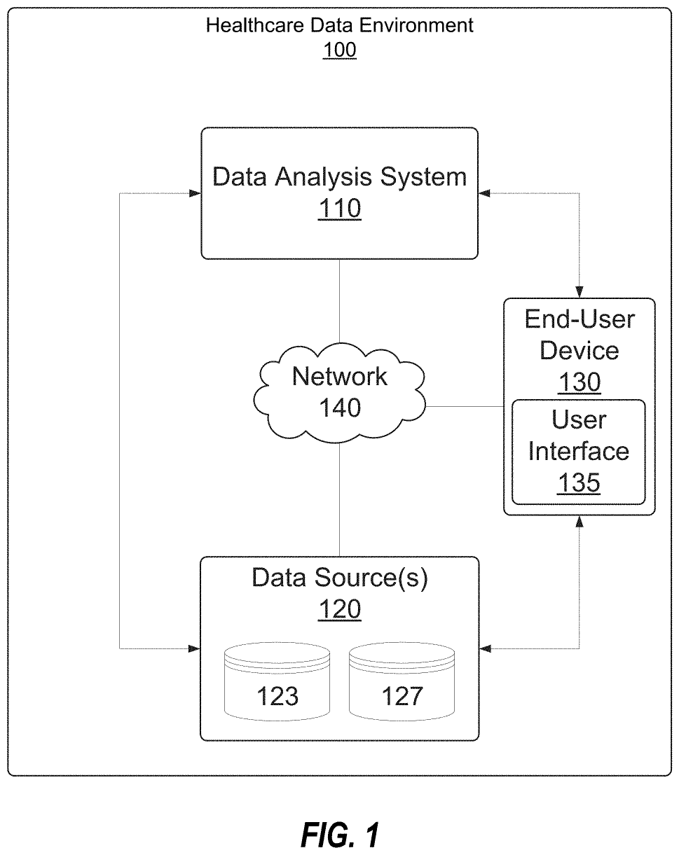

FIG. 1 is an illustration of an exemplary healthcare data environment 100. The healthcare data environment 100 may include a data analysis system 110, one or more data sources 120, and an end-user device 130. A network 140 may connect the data analysis system 110, the one or more data sources 120, and/or the end-user device 130.

The data analysis system 110 may be a computing device, such as a back-end server. The data analysis system 110 may include components that enable data analysis functions and practical applications thereof, such as alerting to inconsistent results of clinical trials through comparison to data stored in EMRs. The data analysis system 110 may use EMRs to conduct virtual clinical trials to study the effects of various ingredients and activities on patient health. The results can be used as source information for clinical trial planning and reporting, or may be used to assess the results of actual clinical trials.

The one or more data sources 120 may be computing devices and/or storage devices configured to supply data to the data analysis system 110. In one example, the one or more data sources 120 comprise a clinical trial database 123, which may be populated with clinical trial information , such as trial results, reports, news sources, medical sources, scientific literature, etc. that report on the results of clinical trials. The clinical trial database 123 may include a clinical trial document, such as a report from a clinical trial from a pharmaceutical company public report or website. The one or more data sources 120 may further include a medical records database 127 storing a plurality of EMRs. In at least some embodiments, the EMRs may provide the data analysis system 110 with information regarding patient medical histories. The EMRs may be enhanced through techniques such as NLP and machine learning classifiers. A system may perform NLP of clinical narrative notes to provide organized data to an EMR from an unstructured format. Applying NLP techniques on clinical narrative notes can extract a variety of measurements, for example, ejection fraction (heart functionality related measure), cancerous tumor dimensions as the disease progresses or regresses over time, body mass index values, severity of a disease (e.g., compensated vs. decompensated chronic obstructive pulmonary disease). Moreover, a classifier developed through machine learning may analyze and EMR to add a medical status or condition to the patient medical history. For instance, a classifier for subjective issues such as pain or diseases that are under-documented such as hypoglycemia may be developed and used to enhance the EMRs. The enhanced EMRs may be stored in the medical records database and used in one or more disclosed methods to alert to results of a virtual clinical trial.

The end-user device 130 may be a computing device (e.g., a desktop or laptop computer, mobile device, etc.). The end-user device 130 may communicate with the data analysis system 110 to receive information and provide feedback related to the evaluation of a clinical trial. In some embodiments, the end-user device 130 may include a user interface 135 enabling a user to view information such as results of a clinical trial and provide input such as select a clinical trial or specific correlation for analysis by the data analysis system 110. In some embodiments, the user interface 135 may be associated with a medical decision support system (MDSS) that provides recommendations regarding treatment options to a clinical user, using results of actual clinical trials and/or virtual clinical trials performed on the basis of EMR data.

The network 140 may be a local or global network and may include wired and/or wireless components and functionality which enable internal and/or external communication for components of the healthcare data environment 100. The network 140 may be embodied by the Internet, provided at least in part via cloud services, and/or may include one or more communication devices or systems which enable data transfer to and from the systems and components of the healthcare data environment 100.

In accordance with some exemplary embodiments, the data analysis system 110, data source(s) 120, end-user device 130, or the related components include logic implemented in specialized hardware, software executed on hardware, or any combination of specialized hardware and software executed on hardware, for implementing the healthcare data environment 100 or related components. In some exemplary embodiments, the data analysis system 110 or any of its components may be or include the IBM Watson™ system available from International Business Machines Corporation of Armonk, N.Y., which is augmented with the mechanisms of the illustrative embodiments described hereafter.

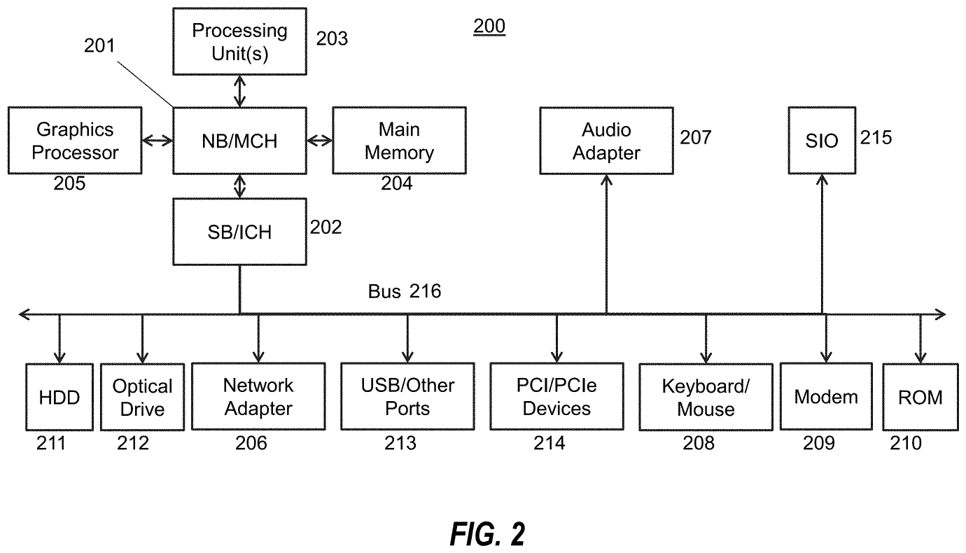

FIG. 2 is a block diagram of an example data processing system 200 in which aspects of the illustrative embodiments are implemented. Data processing system 200 is an example of a computer in which computer usable code or instructions implementing the process for illustrative embodiments of the present invention are located. In one embodiment, FIG. 2 represents the data analysis system 110, which implements at least some of the aspects of the healthcare data environment 100 described herein.

In the depicted example, data processing system 200 can employ a hub architecture including a north bridge and memory controller hub (NB/MCH) 201 and south bridge and input/output (I/O) controller hub (SB/ICH) 202. Processing unit 203, main memory 204, and graphics processor 205 can be connected to the NB/MCH 201. Graphics processor 205 can be connected to the NB/MCH 201 through an accelerated graphics port (AGP).

In the depicted example, the network adapter 206 connects to the SB/ICH 202. The audio adapter 207, keyboard and mouse adapter 208, modem 209, read only memory (ROM) 210, hard disk drive (HDD) 211, optical drive (CD or DVD) 212, universal serial bus (USB) ports and other communication ports 213, and the PCl/PCIe devices 214 can connect to the SB/ICH 202 through bus system 216. PCl/PCIe devices 214 may include Ethernet adapters, add-in cards, and PC cards for notebook computers. ROM 210 may be, for example, a flash basic input/output system (BIOS). The HDD 211 and optical drive 212 can use an integrated drive electronics (IDE) or serial advanced technology attachment (SATA) interface. The super I/O (SIO) device 215 can be connected to the SB/ICH 202.

An operating system can run on processing unit 203. The operating system can coordinate and provide control of various components within the data processing system 200. As a client, the operating system can be a commercially available operating system. An object-oriented programming system, such as the Java™ programming system, may run in conjunction with the operating system and provide calls to the operating system from the object-oriented programs or applications executing on the data processing system 200. As a server, the data processing system 200 can be an IBM® eServer™ System p® running the Advanced Interactive Executive operating system or the Linux operating system. The data processing system 200 can be a symmetric multiprocessor (SMP) system that can include a plurality of processors in the processing unit 203. Alternatively, a single processor system may be employed.

Instructions for the operating system, the object-oriented programming system, and applications or programs are located on storage devices, such as the HDD 211, and are loaded into the main memory 204 for execution by the processing unit 203. The processes for embodiments of the website navigation system can be performed by the processing unit 203 using computer usable program code, which can be located in a memory such as, for example, main memory 204, ROM 210, or in one or more peripheral devices.

A bus system 216 can be comprised of one or more busses. The bus system 216 can be implemented using any type of communication fabric or architecture that can provide for a transfer of data between different components or devices attached to the fabric or architecture. A communication unit such as the modem 209 or network adapter 206 can include one or more devices that can be used to transmit and receive data.

Those of ordinary skill in the art will appreciate that the hardware depicted in FIG. 2 may vary depending on the implementation. For example, the data processing system 200 includes several components which would not be directly included in some embodiments of the data analysis system 110. However, it should be understood that a data analysis system 110 may include one or more of the components and configurations of the data processing system 200 for performing processing methods and steps in accordance with the disclosed embodiments.

Other internal hardware or peripheral devices, such as flash memory, equivalent non-volatile memory, or optical disk drives may be used in addition to or in place of the hardware depicted. Moreover, the data processing system 200 can take the form of any of a number of different data processing systems, including but not limited to, client computing devices, server computing devices, tablet computers, laptop computers, telephone or other communication devices, personal digital assistants, and the like. Essentially, data processing system 200 can be any known or later developed data processing system without architectural limitation.

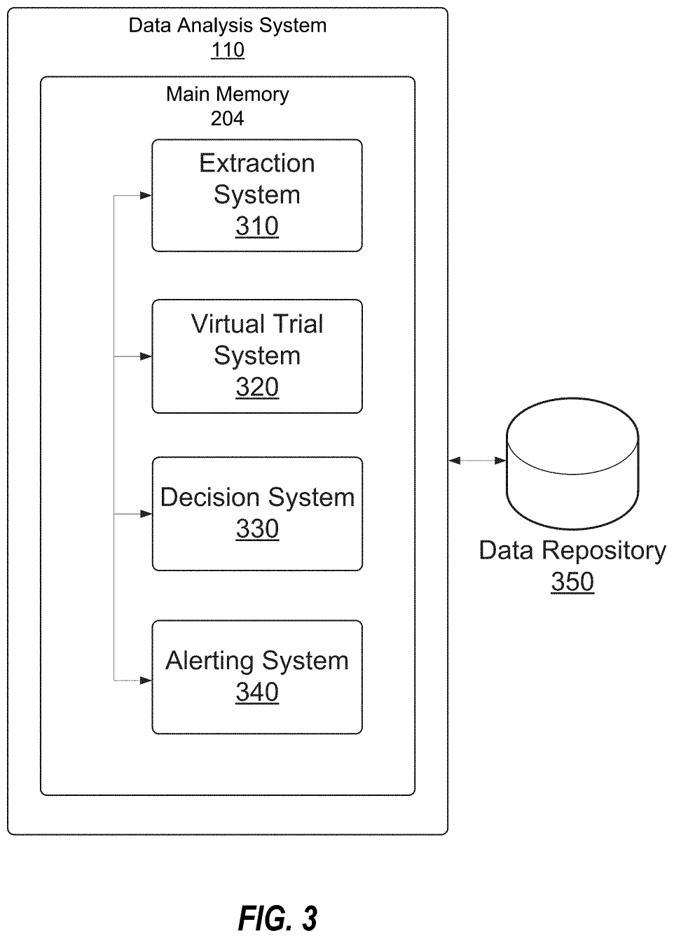

FIG. 3 illustrates an exemplary embodiment of the data analysis system 110. In an exemplary embodiment, the data analysis system 110 includes an extraction system 310, a virtual trial system 320, a decision system 330, and an alerting system 340. These subsystems of the data analysis system 110 may components of a single device, or may be separated devices connected to each other (e.g., via the network 140). In some embodiments, the data analysis system 110 may further include and/or be connected to one or more data repositories 350.

The extraction system 310 may be a computing device or component (e.g., software or hardware engine or module) configured to extract data from the one or more data sources 120. The extraction system 310 may be configured to perform natural language processing on data elements within the one or more data sources 120. The data sources 120 may include data elements such as clinical trial reports, EMRs, and other medical or scientific documents. The extraction system 310 is configured to perform NLP on these and other data elements to extract information that is useful in disclosed processes. The extraction system 310 may be further configured to analyze organized reports and structured data, such as through metadata tags and/or categorization.

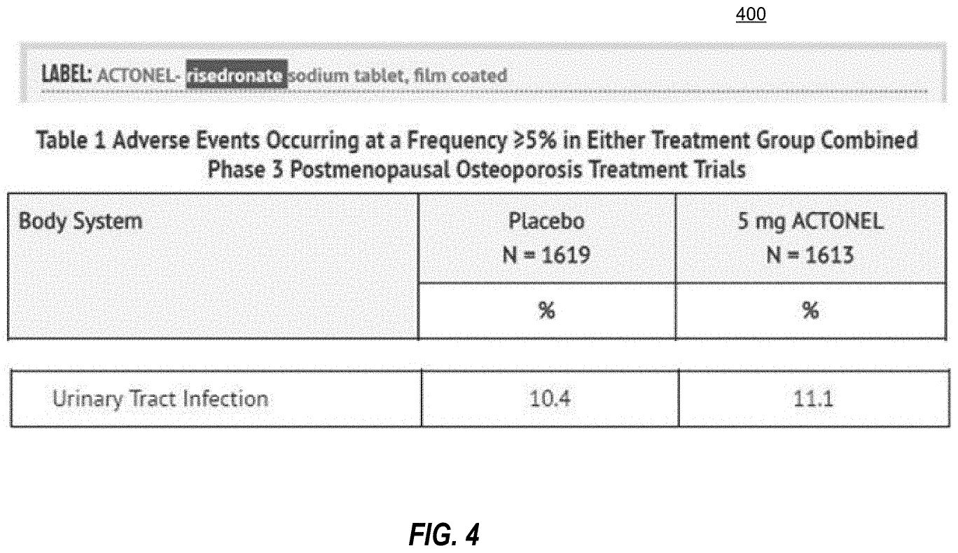

In some embodiments, the extraction system 310 is configured to extract information related to outcomes of one or more clinical trials from clinical trial database 123. For instance, the extraction system 310 may be configured to perform NLP of a clinical trial document, such as the clinical trial report 400 depicted in FIG. 4. In one example, the extraction system uses NLP techniques to extract information from publically-available databases (e.g., Side Effect Resource [SIDER]). The extraction system 310 may be configured to identify results of a clinical trial and information such as ingredient taken and effect of the ingredient determined from the trial. For instance, in a clinical trial, the results generally compare the results of a population that received an ingredient with a group that received only a placebo. In the example of report 400 in FIG. 4, the extraction system 310 may be configured to identify that a clinical trial involving the ingredient risedronate (an osteoporosis drug) reports an increased occurrence of urinary tract infections (UTIs) among those that actually received the ingredient. The extraction system 310 may be configured to review the report 400 and extract the correlation.

The extraction system 310 is further configured to extract information from medical records database 127. For instance, the extraction system 310 may be configured to extract medical history information from a plurality of EMRs. The EMRs may include medical history information that occurs over a period of time such that the effects of certain activities may be determined. For example, the extraction system 310 may use data filters to obtain information associated with patients that satisfy certain criteria in order to extract correlations that, for example, may be compared to the results of one or more clinical trials. In some embodiments, the extraction system 310 is configured to identify covariates that related to a selected outcome. Given a large collection of covariates and an outcome, a covariate selection machine learning algorithm can identify a sub-set of the covariates with the highest level of association relative to the outcome. For example, if the outcome is defined as a new diagnosis of congestive heart failure (CHF) after an exposure to COX-2 inhibitors (an anti-inflammatory pain relief medication), the system is capable of extracting the outcome (date of a diagnosis code or date of a non-negated diagnosis discussed in a note). The system is also capable of extracting a comprehensive list of covariates relative to the exposure date. Covariates may include demographic details, comorbidities, additional medications, and laboratory values. Prior to extraction of the covariates and outcome additional configuration parameters are defined and provided to the system, including length of follow-up window (e.g., CHF is diagnosed within 12 months after the exposure date), and observation window (covariates are extracted during this window before the exposure). A feature selection algorithm can help narrowing down the list of covariates and select the most informative ones. It should be noted that covariate types could be categorial (e.g., gender: male or female) or continuous (e.g., creatinine level). An outcome could be binary (e.g., patient develops CHF during the follow-up window, or no indication for the disease was identified). An outcome could be also multinomial with several possible values; for example, an outcome with the following possibilities: 1) no heart failure, 2) heart failure with preserved ejection fraction, 3) heart failure with reduced ejection fraction, and 4) heart failure with an unknown type.

The virtual trial system 320 may be a computing device or component (e.g., software or hardware engine or module) configured to build an association model to perform a virtual clinical trial using patient history data from EMRs. The virtual trial system 320 is configured to perform logistical analysis on extracted EMR data to assess the level of association between taking one or more ingredients and resulting effects (e.g., development of a disease, pain, headaches, fatigue, etc.). The virtual trial system 320 is configured to apply computational algorithms to assess the effects of each ingredient separately or a combination of ingredients. An example of a methodology to assess the level of association between taking an ingredient and a certain medical outcome relies on identifying populations of cases and controls found in the EMR database (often denoted as “exposed” and “unexposed”, respectively). Cases are patients who received the treatment and controls are patients who did not receive the treatment. Examples of computational approaches to identify matched cases and controls include an exact match (e.g., a case and a control have the same gender and race), and distance-based match in which a variety of covariates are considered and balanced. Propensity score matching, for example, could be used as an approach to filter out cases and controls that may increase a bias of identifying an accurate treatment effect.

The virtual trial system 320 may be configured to search medical history information of a plurality of patients to find exposed and unexposed populations that relate to one or more covariates and dependent variables, such as a the taking of an ingredient (e.g., risedronate) and an outcome (e.g., developing a subsequent UTI). The exposed population includes patients that have taken the ingredient and match other criteria (e.g., age, gender, disease, condition, etc.) and the unexposed population includes patients that have not taken the ingredient and match the other criteria. The exposed and unexposed populations then can be considered as subjects of the virtual clinical trial, with the exposed population being the group that has taken the ingredient and the unexposed population being the group that has taken a placebo.

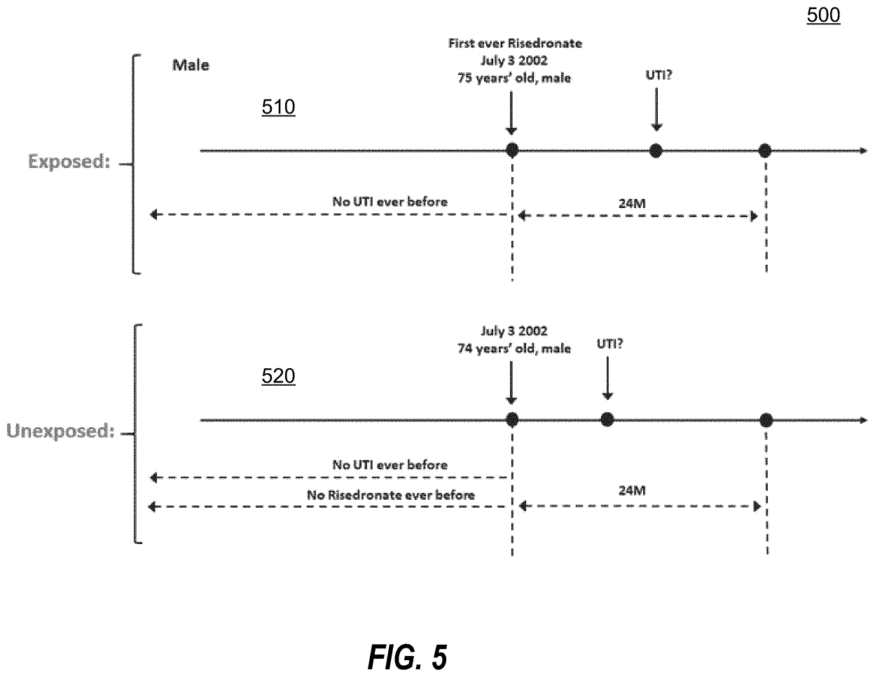

FIG. 5 is a depiction of examples of medical history information 500 for two patients, 510, 520 from EMR data that may be selected by the virtual trial system 320. Patient 510 may be an example from the exposed population found in the EMR data. Patient 520 may be an example from the unexposed population found in the EMR data. As described above, a variety of computational methods could be used to identify exposed and match unexposed populations; often the methods may also identify more than one matched unexposed individual for a given exposed individual. Additional factors that could be used to match exposed and unexposed populations may include genetics-related variables, as well as variables captured by using wearable devices. As described herein, the EMR data may be enhanced through techniques including NLP and machine learning classifiers. For instance, the analysis system 110 may perform NLP of a clinical narrative note to determine that patient 510 was written a prescription for risedronate and/or that patient 520 experienced a UTI, for example. In another example, the EMR data may be enhanced with through a machine learning classifier that determines a subjective effect, such as pain or a condition such as a compensated or decompensated status of congestive heart failure. The virtual trial system 320 may use the enhanced EMR data to perform a virtual clinical trial.

The virtual trial system 320 may be configured to execute computational algorithms such as propensity score matching, logistic regression, and bootstrapping to determine factors such as an odds ratio and/or p-value that indicates the outcome of the virtual trial. For example, the analysis of EMR data may determine that there is only a small correlation between risedronate and development of a UTI.

The decision system 330 may be a computing device (e.g., software or hardware engine or module) configured to compare results of actual clinical trial data from the extraction system 310 and virtual clinical trial data from the virtual trial system 320. For example, the decision system 330 may receive a correlation from the extraction system 310 (e.g., a result that indicates a certain ingredient produces a certain adverse effect) and statistical data from the virtual trial system (e.g., present odds ratio and/or present p-value) and compare the information to determine an accuracy score. For example, the decision system 330 may determine that a determined effect from a clinical trial is validated or contradicted and store the result in the one or more data sources 120. Examples of accuracy scores that the decision system 330 could produce include, for example: validated, contradicted, partially validated, partially contradicted, supported, unsupported, insufficient data, etc. In another example, the decision system 330 may provide a numerical rating on an accuracy scale (e.g., percentage from 0-100%) that assesses the accuracy of a selected correlation.

The alerting system 340 may be a computing device (e.g., software or hardware engine or module) configured to provide an alert based on a result from the decision system 330. The alerting system 340 may provide an accuracy score for a selected correlation from a clinical trial. For example, the alerting system 340 may provide an alert to the end-user device 130 when a virtual clinical trial produces a result that contradicts a result from an actual clinical trial. In another example, the alerting system 340 may be a communication module configured to provide requested results of a virtual clinical trial when requested by a user through interaction with the user interface 135.

The data repository 350 may be a database configured to store data. The data repository 350 may be configured to receive data from the extraction system 310 and/or from one or more data sources 120 and store the data according to appropriate storage protocols. In some embodiments, the data repository 350 receives data from the data analysis system 110, such as from the extraction system 310. In other embodiments, the data repository 350 receives data from the one or more data sources 120 and is a data supply for the data analysis system 110.

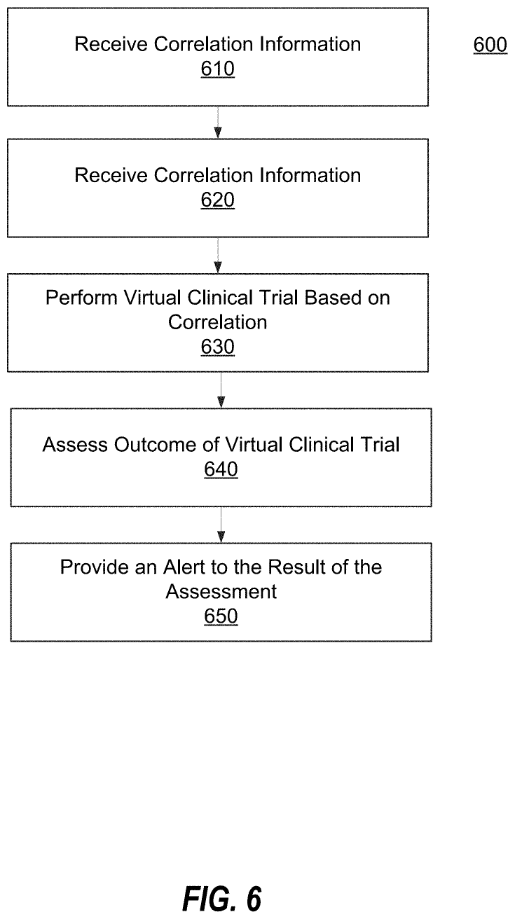

FIG. 6 is a flowchart of an exemplary process for alerting to an assessment of a result from a virtual clinical trial. The data analysis system 110 may perform one or more steps of the process 600 in order to use information from data source(s) 120 (e.g., EMR data) to determine whether stored medical history information provides statistically relevant information regarding a target correlation.

In step 610, the data analysis system 110 receives information regarding a correlation. A correlation may include, in general, a target cause and target effect. The target cause may include, for example, the taking of one or more ingredients. The target effect may include, for example, a medical status or condition of a patient. For example, the extraction system 310 may receive a target correlation for analysis.

In one example, the extraction system 310 receives a request from the end-user device 130 to review a correlation. For instance, a user may review a table of results from a clinical trial and identify a correlation that indicates that a certain ingredient resulted in the exposed population having an increased occurrence of an adverse condition (e.g., development of a UTI, high blood pressure, congestive heart failure, death, etc.). The user may provide input to the end-user device 130 that causes the end-user device to deliver the target correlation to the extraction system 310.

In another example, the extraction system 310 may extract information from a clinical trial document, such as by using NLP, to identify a correlation from the document. For example, the extraction system 310 may process a data table stored in the clinical trial database 123 and select a correlation.

In step 620, the data analysis system 110 receives EMR data. For example, the extraction system 310 may extract medical history information form the medical records database 127. The extraction system 310 may identify one or more independent variable/cause (e.g., the ingredient/treatment taken) and a dependent variable/result (e.g., the medical condition) from the target correlation and use this information to extract relevant EMR data. The extraction system 310 may use additional filtering of data such as age, gender, etc. in order to narrow the extracted data to relevant patients. In one example, the extraction system 310 may extract information for an exposed population and an unexposed population based on the ingredient taken; an exposed patient was identified with a prescription of the ingredient and an unexposed patient had no indications for taking the ingredient. In some embodiments, only an independent variable and filter data is used to identify exposed and unexposed populations, with all or a set of effects/dependent variables being identified from the data.

In step 630, the data analysis system 110 performs an analysis, such as a virtual clinical trial, based on the extracted EMR data. For example, the virtual trial system 320 to determine one or more accuracy scores for the target correlation. In one example, the virtual trial system 320 calculates a present odds ratio and present p-value based on the exposed unexposed populations received by the extraction system 310.

In one example, the virtual trial system 320 uses computational algorithms to perform a statistical analysis on the extracted EMR data. For example, the virtual trial system 320 may perform propensity score matching, logistic regression, and/or bootstrapping techniques applied on a set of matched exposed-unexposed individuals to determine the present odds ratio and present p-value. Additional examples of methods to estimate the level of association between taking an ingredient and an outcome include, for example “approximate adjustment” (Zhang J, Yu K F. What's the relative risk? A method of correcting the odds ratio in cohort studies of common outcomes. JAMA 1998;280:1690-1), “relative risk regression” (Barros A J, Hirakata V N. Alternatives for logistic regression in cross-sectional studies: an empirical comparison of models that directly estimate the prevalence ratio. BMC Med Res Methodol 2003;3:21), and “risk ratio” (Ospina P A, Nydam D V, DiCiccio T J. Technical note: The risk ratio, an alternative to the odds ratio for estimating the association between multiple risk factors and a dichotomous outcome. J Dairy Sci. 2012 May;95(5):2576-84).

In some embodiments, the virtual trial system 320 may also provide one or more unreported correlations based on the statistical analysis. For example, the virtual trial system 320 may analyze the exposed and unexposed populations to identify effects that are not associated with a target correlation. In one example, the clinical trial data may only include an ingredient and some limiting factors and all results may be considered unreported correlations. In another example, the clinical trial data may include an effect and the virtual trial system 320 may perform a virtual clinical trial and identify a different effect that is not otherwise provided. For instance, a request to assess a correlation between risedronate and the development of a UTI may result in a determination the risedronate is associated with an increase likelihood for CHF based on EMR data.

In step 640, the data analysis system 110 assesses the outcome of the virtual clinical trial. For example, the decision system 330 may determine an accuracy score of a target correlation, the accuracy score indicating whether the proposed effect is actually associated by the target cause. For example, the accuracy score may indicate that a target correlation is verified or unverified, supported or unsupported, or unknown. The decision system 330 may compare the odds ratio and p-value determine by the virtual trial system to the actual clinical trial results to detect discrepancies or accurate results.

In step 650, the data analysis system 110 may provide an alert based on the result of the decision system 330. For example, the alerting system 340 may provide an alert to a target correlation selected from an actual clinical trial that is contradicted by a virtual clinical trial. In another example, the alerting system 340 may provide the accuracy score for a target correlation that is received from end-user device 130. For example, in one exemplary process, a user may input a correlation to end-user device 130 (e.g., through user interface 135) which is received by the data analysis system 110. The data analysis system 110 performs process 600 and provides a result back to the end-user device 130 (e.g., through user interface 135).

The process 600 provides an assessment process for checking the results of clinical trials, as well as determining areas of interest for future trials. For instance, the process 600 may be used to identify correlations in EMR data that appear to be accurate exposure-effect associations. These results may be tested further through clinical trials. Moreover, the ability of the data analysis system 110 to perform NLP of clinical trial documents provides an automated system for leveraging data in EMRs to provide automatic assessments of the results.

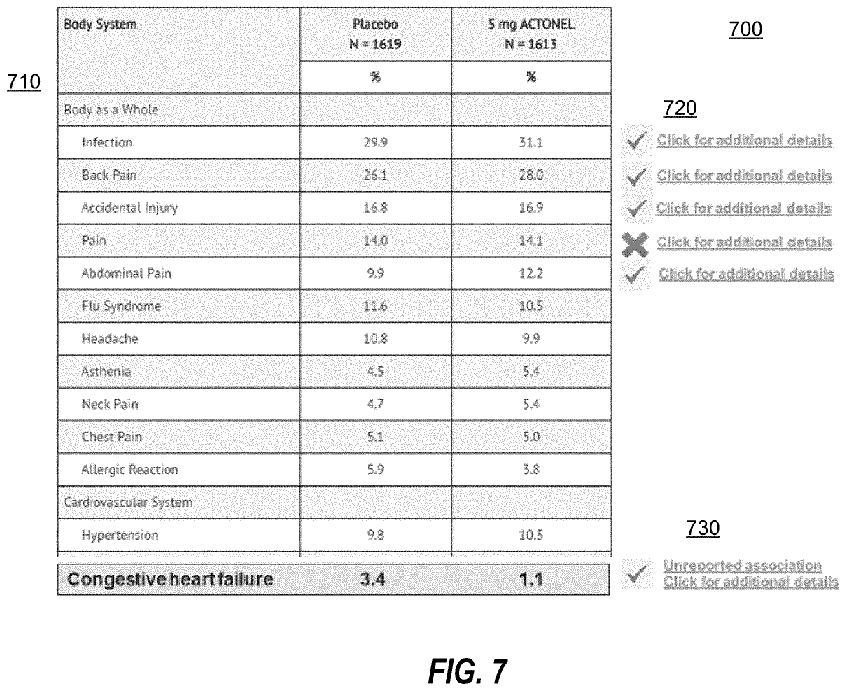

FIG. 7 is an example of a user interface 700 that may be displayed using end-user device 130. The user interface 700 includes a table 710 providing results of a clinical trial. The table 710 includes a comparison of occurrence rates for exposed and unexposed populations. The user interface 700 further includes a visual indicator 720 indicating the result of a virtual clinical trial for each correlation in the table 710. For example, a “checkmark” may be presented for verified correlations while an “X” may be presented for contradicted correlations. The data analysis system 110 may be configured to automatically extract each correlation in the table 710, perform a virtual trial process, and provide the results to the user interface 700 for displaying to a user. In some embodiment, the data analysis system 110 may also provide one or more unreported correlations 730 to the user interface 700, which may be effects that are found in the EMR data which were not originally present in the table 710. In this way, the data analysis system 110 may enhance clinical trial results through the additional analysis of a virtual clinical trial.

The present description and claims may make use of the terms “a,” “at least one of,” and “one or more of,” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the example provided herein without departing from the spirit and scope of the present invention.

The system and processes of the Figures are not exclusive. Other systems, processes and menus may be derived in accordance with the principles of embodiments described herein to accomplish the same objectives. It is to be understood that the embodiments and variations shown and described herein are for illustration purposes only. Modifications to the current design may be implemented by those skilled in the art, without departing from the scope of the embodiments. As described herein, the various systems, subsystems, agents, managers, and processes can be implemented using hardware components, software components, and/or combinations thereof. No claim element herein is to be construed under the provisions of 35 U.S.C. 112, sixth paragraph, unless the element is expressly recited using the phrase “means for.”

Although the invention has been described with reference to exemplary embodiments, it is not limited thereto. Those skilled in the art will appreciate that numerous changes and modifications may be made to the preferred embodiments of the invention and that such changes and modifications may be made without departing from the true spirit of the invention. It is therefore intended that the appended claims be construed to cover all such equivalent variations as fall within the true spirit and scope of the invention.

What is claimed is:

1. A computer-implemented method for performing a virtual clinical trial and alerting to results in a data processing system comprising a processing device and a memory comprising instructions which are executed by the processor, the method comprising:
extracting data from a clinical trial document, the clinical trial document including results of a clinical trial and including an ingredient;
extracting data from a medical records database based on the ingredient, the data including medical history information for a plurality of patients;
performing a virtual clinical trial on the medical history information from the plurality of patients;
assessing the results of the virtual clinical trial, including determining a result associated with a correlation involving the ingredient; and
alerting to the result by providing information to a user interface of an end-user device.

2. The method of claim 1, wherein extracting data from the clinical trial document comprises performing natural language processing.

3. The method of claim 2, wherein the clinical trial document includes correlations between the ingredient and one or more effects of the ingredient.

4. The method of claim 3, wherein the clinical trial document includes effects for a population that received the ingredient and a population that received a placebo.

5. The method of claim 1, wherein extracting the data from the medical records database comprises performing natural language processing of clinical narrative notes to obtain the medical history information.

6. The method of claim 1, wherein extracting the data from the medical records database further comprises identifying a first population of patients having a medical history information indicating they were exposed to the ingredient and a second population of patients having a medical history information indicating they were unexposed to the ingredient.

7. The method of claim 6, wherein identifying the first and second populations of patients comprises limiting the results based on patient criteria.

8. The method of claim 7, wherein the patient criteria include age, gender, or medical condition.

9. The method of claim 5, wherein performing a virtual clinical trial comprises performing statistical analysis of the first and second populations.

10. The method of claim 9, wherein performing the statistical analysis comprises determining one or more of a present odds ratio or a present p-value.

11. The method of claim 10, wherein the result associated with the correlation comprises the odds ratio or the present p-value.

12. The method of claim 9, wherein result associated with the correlation comprises an accuracy score.

13. A data analysis system for performing a virtual clinical trial and alerting to results, comprising:
an extraction system configured to:
extract a correlation between an ingredient and an effect from a clinical trial document using natural language processing, and
extract medical history information for a population exposed to the ingredient and a population unexposed to the ingredient from stored electronic medical records;
a virtual trial system configured to perform statistical analysis on the medical history information to assess the level of association between the ingredient and the effect;
a decision system configured to determine an accuracy score for the correlation based on the statistical analysis; and
an alerting system configured to provide the accuracy score to a user interface of an end-user device.

14. The data analysis system of claim 13, wherein the clinical trial document includes a table indicating the effect on a population that received the ingredient and a population that received a placebo.

15. The data analysis system of claim 13, wherein the statistical analysis comprises propensity score matching and logistic regression to determine one or more of a present odds ratio and a present p-value.

16. The data analysis system of claim 15, wherein the logistic regression is adjusted for patient criteria comprising one or more of age, gender, or ethnicity.

17. The data analysis system of claim 13, wherein the accuracy score comprises a label indicating the accuracy of the correlation identified in the clinical trial document.

18. The data analysis system of claim 13, wherein the alerting system is configured to provide a visual indicator to the user interface to represent the accuracy score.

19. The data analysis system of claim 13, wherein the virtual trial system is further configured to identify a correlation between the ingredient and a second effect as an unreported association in the clinical trial document and the alerting system is configured to provide the unreported association to the user interface.

20. A computer program product for performing a virtual clinical trial and alerting to results, the computer program product comprising a computer readable storage medium having program instructions embodied therewith, the program instructions executable by a processor to cause the processor to:
extract data from a clinical trial document, the clinical trial document including results of a clinical trial and including an ingredient;
extract data from a medical records database based on the ingredient, the data including medical history information for a plurality of patients;
perform a virtual clinical trial on the medical history information from the plurality of patients;
assess the results of the virtual clinical trial, including determining a result associated with a correlation involving the ingredient; and
alert to the result by providing information to a user interface of an end-user device.
